(19) United States
(12) Patent Application Publication
(10) Pub. No.: US 2026/0090814 A1
(43) Pub. Date: Apr. 2, 2026

(54) ADJUSTABLE NAVIGATED SURGICAL DRILL SHEATH

(71) Applicant: Medivis, Inc., New York, NY (US)

(72) Inventors: Jesse Harrison Gallant, New York, NY (US); Osamah Choudhry, New York, NY (US); Christopher Morley, New York, NY (US); Florentin Jonas Liebmann, New York, NY (US)

(21) Appl. No.: 18/900,135

(22) Filed: Sep. 27, 2024

(51) Int. Cl. A61B 17/17; A61B 17/00

(52) U.S. Cl. CPC A61B 17/17; A61B 2017/0046

(57) ABSTRACT

An adjustable navigated surgical drill sheath includes a handle, main body, and adjustment arrangement. The main body can receive a separate surgical drill bit through a top opening, a hollow region therethrough, and a bottom opening. The adjustment arrangement can facilitate setting a specific drill depth corresponding to the maximum distance that the drill bit can extend past the bottom opening and allows for multiple different specific drill depths. The adjustment arrangement can include a removable insert having locking features than can be moved up and down within the hollow region and a latching mechanism that interacts with the locking features to lock the insert at a specific position relative to the main body to set the specific drill depth. Fiducial markers coupled to the main body can facilitate use of the surgical drill sheath as a navigated device in an augmented reality environment.

### TECHNICAL FIELD

The present disclosure relates generally to medical devices, and more particularly to tools and devices used during medical procedures.

### BACKGROUND

Planning and navigation are necessary for many medical procedures, such as live surgeries, practices, training, planning, scanning, and the like. Surgical teams typically have a plan based on medical imagery before ever entering an operating room. Conventional medical imaging systems such as X-ray, MRI, CT, and others have limitations regarding two-dimensional and three-dimensional images, however, and surgeons often need to consider numerous image views and slices to plan surgical procedures. Recent medical advances leverage these applications of medical imagery and surgical plans by using a computer-aided augmented reality environment, which can allow for the tracking of patients and physical instruments during surgical procedures by using reference (i.e., fiducial) markers and associated tracking components. Other medical procedures, such as ultrasound scanning, have also made use of computer-aided augmented reality environments involving tracked reference markers.

Unfortunately, conventional tracking systems are often limited in their ability to accurately generate, render, and apply virtual interactions in an augmented reality environment based on the orientations and positions of physical instruments with respect to those of physical landmarks identified on a patient body or other relevant location, particularly when things move during surgery or another relevant medical procedure. Unstable or unreliable positioning of fiducial markers can play a role in these issues. Limited or inaccurate tracking can affect the overall performance of such systems during surgeries or other relevant medical procedures. This can be particularly true for medical devices and physical instruments that include components or features that are not readily tracked, such as those that are located within a patient body during a surgery or other medical procedure.

One example of a medical device that can be tracked in an augmented reality environment is a surgical drill. Multiple reference markers coupled to or about a surgical drill body can be used to track the location of the drill body and also the drill bit while the drill bit remains outside of a patient body. When the surgical drill bit is inserted into a patient body and operated, however, the reference markers are typically unhelpful for determining the exact depth that the drill bit has traveled. In the case of a pedicle drill, for example, a surgeon operating the drill can utilize the reference markers and augmented reality system to position the drill bit at a proper location outside the patient body, but then must rely on technique and skill in operating the pedicle drill to drill a hole in the patient pedicle at about a desired depth. Some amount of drill depth error may occur, as the hole often cannot be drilled to a desired depth exactly due to the drill bit tip being inside the patient and not being visible or trackable in augmented reality. This inexact drill depth issue can be compounded in situations where multiple holes of different sizes and/or depths are to be drilled during a given surgery or procedure. Inexact drilled depths can also be a concern for other surgical drills besides pedicle drills.

While traditional ways of operating a surgical drill have worked well in the past, improvements are always helpful. In particular, what is desired are devices and methods that allow surgical drills to be operated to drill holes within a patient to exact drill depths with minimal to no error, even where multiple drilled holes of different depths are desired.

### SUMMARY

It is an advantage of the present disclosure to provide medical systems, devices, and methods that allow surgical drills to be operated to drill holes within a patient to exact depths with minimal to no error, even where multiple drilled holes of different depths are desired. The disclosed embodiments relate to surgical drill sheaths that can be used to guide surgical drill bits during surgeries and other medical procedures. In particular, the disclosed systems, devices, and methods can involve adjustable navigated surgical drill sheaths configured for use with surgical drills, with the drill sheath having adjustment arrangements such that the surgical drills can be operated to drill holes within patients to exact depths.

In various embodiments of the present disclosure, a surgical drill sheath can include at least a handle, a main body, and an adjustment arrangement. The handle can be configured to allow an operator to hold the surgical drill sheath. The main body can be coupled to the handle and can include a top region, a bottom region, and a hollow region between the top and bottom regions. The main body can be configured to receive a separate surgical drill bit through a top opening in the top region, through the hollow region, and through a bottom opening in the bottom region. The adjustment arrangement can be configured to facilitate setting a specific drill depth for the separate surgical drill bit received through the main body. The specific drill depth can correspond to the maximum distance that the separate surgical drill bit can extend past the bottom opening of the main body. The adjustment arrangement can allow for a plurality of different specific drill depths.

In various detailed embodiments, the adjustment mechanism can include a mechanical hard stop proximate the top region, with this mechanical hard stop being configured to abut against a bottom surface of a separate surgical drill operating the separate surgical drill bit when the specific drill depth is reached. The adjustment mechanism can be configured to facilitate adjusting a distance between the mechanical hard stop and the bottom opening. In some arrangements, the separate surgical drill can be a pedicle drill. The adjustment mechanism can include a movable drill sheath insert located within the hollow region and configured to be moved up and down within the hollow region to facilitate setting the specific drill depth. This movable drill sheath insert can include a plurality of locking features along an outer surface thereof, and the adjustment mechanism can further include a latching mechanism configured to interact with the plurality of locking features to lock the movable drill sheath insert at a specific locked position relative to the main body to facilitate setting the specific drill depth. The latching mechanism can include a pivoting member having a pawl that engages with the plurality of locking features to lock the movable drill sheath insert at the specific locked position. The pivoting member can be spring loaded to lock the movable drill sheath insert at a specific locked position, and the pivoting member can further include a manual release feature to overcome the spring loaded locked position and allow the movable drill sheath insert to be moved.

In further detailed embodiments, the movable drill sheath insert can include a cylindrical opening therethrough configured to accept the separate surgical drill bit therethrough and having a diameter that corresponds to the diameter of the separate surgical drill bit. The movable drill sheath insert can be configured to be removed from the main body. The surgical drill sheath can also be configured to receive an alternative movable drill sheath insert within the main body when the movable drill sheath insert is removed. The movable drill sheath insert, and alternative movable drill sheath insert can be configured for use with differently dimensioned separate surgical drill bits. In some arrangements, the surgical drill sheath can be configured for use with separate surgical drill bits having diameters of about 2, 2.5, or 3 mm. The plurality of different specific drill depths can range from about 8 to 35 mm. The plurality of different specific drill depths can be adjusted by increments of about 2 mm. In some arrangements, the surgical drill sheath can also include a plurality of fiducial markers coupled to the main body, which plurality of fiducial markers can be configured to facilitate use of the surgical drill sheath as a navigated device in an augmented reality environment.

In further embodiments of the present disclosure, an adjustable navigated surgical drill sheath can include a handle, a main body, an adjustment arrangement, and a plurality of fiducial markers. The handle can be configured to allow an operator to hold the adjustable navigated surgical drill sheath. The main body can be coupled to the handle and can include a top region, a bottom region, and a hollow region between the top and bottom regions. The main body can be configured to receive a separate surgical drill bit through a top opening in the top region, through the hollow region, and through a bottom opening in the bottom region. The adjustment arrangement can be configured to facilitate setting a specific drill depth for the separate surgical drill bit received through the main body, and this specific drill depth can correspond to the maximum distance that the separate surgical drill bit can extend past the bottom opening. The adjustment arrangement can allow for a plurality of different specific drill depths and can include a removable drill sheath insert and a latching mechanism. The removable drill sheath insert can have a plurality of locking features and can be configured to be moved up and down within the hollow region. The latching mechanism can have a spring loaded pivoting member with a pawl configured to interact with the plurality of locking features to lock the removable drill sheath insert at a specific locked position relative to the main body to facilitate setting the specific drill depth. The plurality of fiducial markers can be coupled to the main body and can be configured to facilitate use of the surgical drill sheath as a navigated device in an augmented reality environment.

In various detailed embodiments, the removable drill sheath insert can include a cylindrical opening therethrough having a diameter that corresponds to the separate surgical drill bit diameter. The removable drill sheath insert can be configured to be replaced with an alternative removable drill sheath insert having a cylindrical opening of a different diameter that corresponds to a separate alternative surgical drill bit diameter. In various arrangements, the adjustable navigated surgical drill sheath can be configured for use with separate pedicle drill bits having diameters of about 2, 2.5, or 3 mm. The plurality of different specific drill depths can range from about 8 to 35 mm, and the plurality of different specific drill depths can be adjusted by increments of about 2 mm.

In still further embodiments of the present disclosure, various methods of using a surgical drill sheath are provided. Pertinent process steps can include placing a drill sheath insert, adjusting the drill sheath insert, and guiding a separate surgical drill bit. The drill sheath insert can be placed into a main body of the surgical drill sheath, with the main body having a top region, a bottom region, and a hollow region between the top and bottom regions. The drill sheath insert can be adjusted to a correct height within the main body, and the correct height can correspond to a specific drill depth that reflects the maximum distance that a separate surgical drill bit operated by a separate surgical drill can extend past a bottom opening in the bottom region of the main body. The separate surgical drill bit can be guided through the drill sheath insert and the main body of the surgical drill sheath such that the surgical drill bit extends through the entire drill sheath insert as well as through a top opening in the top region, through the hollow region, and through the bottom opening in the bottom region of the main body.

In various detailed embodiments, the separate surgical drill can be a pedicle drill. Further process steps can include choosing a desired length and diameter for one or more pedicle screws to be used in a subsequent pedicle operation, removing an existing drill sheath insert from the main body of the surgical drill sheath, selecting the drill sheath insert to be placed into the main body based on the chosen pedicle screw diameter, contacting a drill head of the pedicle drill against a top of the drill sheath insert adjusted within the main body, and actuating the pedicle drill. The pedicle drill can be actuated until the drill head contacts the top of the drill sheath insert, which can correspond to the drill bit reaching the specific drill depth.

Other apparatuses, methods, features, and advantages of the disclosure will be or will become apparent to one with skill in the art upon examination of the following figures and detailed description. It is intended that all such additional apparatuses, methods, features and advantages be included within this description, be within the scope of the disclosure, and be protected by the accompanying claims.

### BRIEF DESCRIPTION OF THE DRAWINGS

The included drawings are for illustrative purposes and serve only to provide examples of possible structures, arrangements, and methods of use for surgical drill sheaths, such as adjustable navigated surgical drill sheaths. These drawings in no way limit any changes in form and detail that may be made to the disclosure by one skilled in the art without departing from the spirit and scope of the disclosure.

FIG. 1A illustrates in top perspective view an example surgical system with a surgical drill and a surgical drill sheath according to one embodiment of the present disclosure.

FIG. 1B illustrates in bottom perspective view the surgical system of FIG. 1A according to one embodiment of the present disclosure.

FIG. 2A illustrates in side elevation view an example adjustable surgical drill sheath with a raised insert according to one embodiment of the present disclosure.

FIG. 2B illustrates in side elevation view the adjustable surgical drill sheath of FIG. 2A with its insert lowered according to one embodiment of the present disclosure.

FIG. 3 illustrates a flowchart of an example summary method of using a surgical drill sheath according to one embodiment of the present disclosure.

FIG. 4A illustrates in side cross-section view an adjustable region of an example adjustable surgical drill sheath according to one embodiment of the present disclosure.

FIG. 4B illustrates in side cross-section view the adjustable region of FIG. 4A with the insert raised and unlocked according to one embodiment of the present disclosure.

FIG. 4C illustrates in side cross-section view the adjustable surgical drill sheath of FIG. 4A with its insert raised and locked according to one embodiment of the present disclosure.

FIG. 5A illustrates in side elevation view an example adjustable navigated surgical drill sheath according to one embodiment of the present disclosure.

FIG. 5B illustrates in reverse side elevation view the adjustable navigated surgical drill sheath of FIG. 5A according to one embodiment of the present disclosure.

FIG. 5C illustrates in top plan view the adjustable navigated surgical drill sheath of FIG. 5A according to one embodiment of the present disclosure.

FIG. 6 illustrates a flowchart of an example detailed method of using a surgical drill sheath according to one embodiment of the present disclosure.

### DETAILED DESCRIPTION

Exemplary applications of apparatuses, systems, and methods according to the present disclosure are described in this section. These examples are being provided solely to add context and aid in the understanding of the disclosure. It will thus be apparent to one skilled in the art that the present disclosure may be practiced without some or all of these specific details provided herein. In some instances, well known process steps have not been described in detail in order to avoid unnecessarily obscuring the present disclosure. Other applications are possible, such that the following examples should not be taken as limiting. In the following detailed description, references are made to the accompanying drawings, which form a part of the description and in which are shown, by way of illustration, specific embodiments of the present disclosure. Although these embodiments are described in sufficient detail to enable one skilled in the art to practice the disclosure, it is understood that these examples are not limiting, such that other embodiments may be used, and changes may be made without departing from the spirit and scope of the disclosure.

As is generally well known, modern surgeries and other medical procedures are sometimes facilitated by using a computer-aided augmented reality environment. Fiducial markers can be used for tracking patients, medical devices, or other physical instruments during surgeries or other medical procedures. Such fiducial markers can be part of overall systems that can include specialized lighting arrangements, cameras, and computing systems. Attachment devices are often used to locate the fiducial markers in place relative to the patient or other physical device or instrument, and unstable or unreliable positioning of these markers can result in the reduced effectiveness of the overall computer-aided augmented reality environment. While fiducial marker arrangements can be used with surgical drills to track location and orientation of the drill bodies and drill bits outside a patient, additional components can be used to facilitate drilling holes within a patient to exact drill depths.

The present disclosure generally relates to surgical drill sheaths configured to receive surgical drill bits therethrough to guide the surgical drill bits for drilling operations within patients using surgical drills. The disclosed surgical drill sheaths can include adjustment arrangements configured to facilitate setting specific drill depths for the surgical drill bits received through the main body such that the surgical drills can be operated to drill holes within a patient to exact depths with minimal to no error. In specific embodiments, the adjustment mechanisms can involve the use of removable drill sheath inserts within the drill sheath bodies, and each drill sheath insert can be raised or lowered to a locked position corresponding to a desired drill hole depth. A mechanical hard stop at the top of a drill sheath insert can abut against a bottom surface of a separate surgical drill when the specific drill depth is reached.

Although various embodiments disclosed herein discuss pedicle drills and pedicle drilling operations in conjunction with the disclosed surgical drill sheaths, it will be readily appreciated that the disclosed features, apparatuses, systems, and methods can also be used in conjunction with other types of surgical drills and other medical equipment. While various embodiments include the use of fiducial markers to result in the creation of a navigated surgical drill sheath, it will be understood that the disclosed surgical drill sheaths can also be used without fiducial markers and without augmented reality environments in some arrangements. Furthermore, while the disclosed surgical drill sheaths can be used specifically with surgical drills during surgeries or other medical procedures, it is contemplated that these drill sheaths can also be used with non-medical drills or other tools or objects. Other applications, arrangements, and extrapolations beyond the illustrated embodiments are also contemplated.

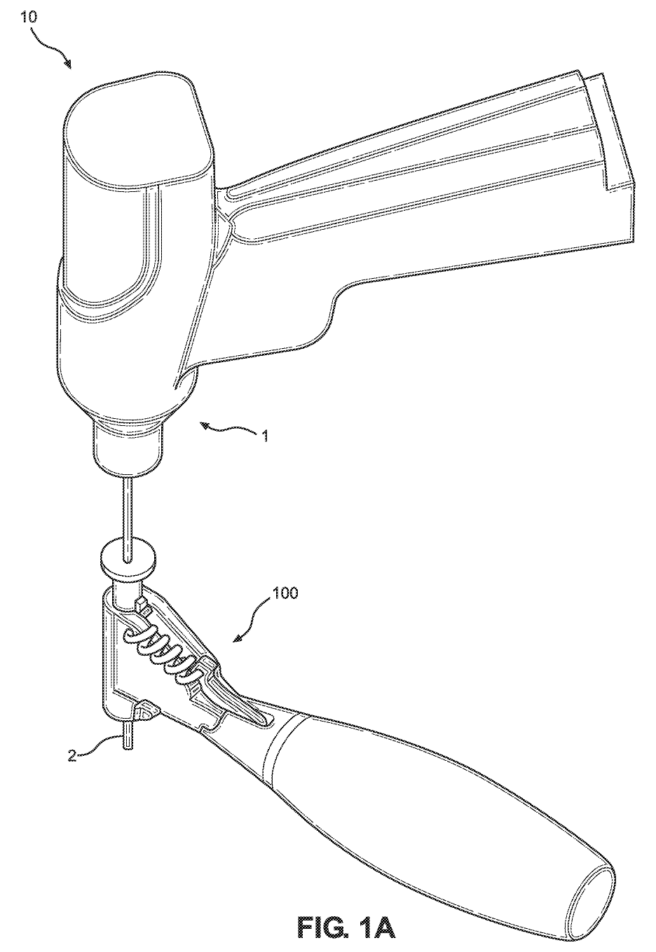

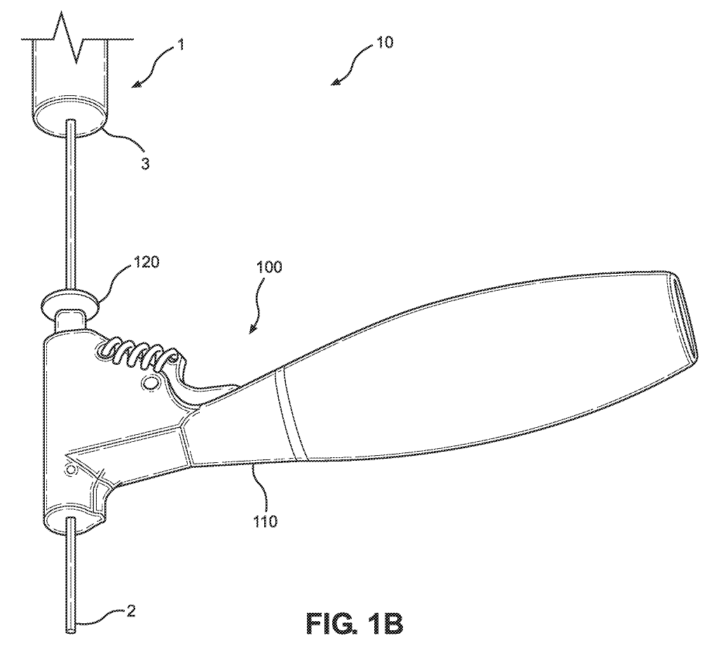

Referring first to FIGS. 1A and 1B, an example surgical system with a surgical drill and surgical drill sheath is illustrated in top and bottom perspective views respectively. In general, surgical system 10 can be configured to facilitate drilling holes into a patient during a surgery or other medical procedure. Surgical system 10 can generally include a surgical drill 1 and a surgical drill sheath 100 arranged for this purpose. Surgical drill 1 can be any suitable drill, such as a pedicle drill, for example, and can include a main body and a narrow elongated surgical drill bit 2 extending therefrom. Surgical drill sheath 100 can be configured to receive surgical drill bit 2 therethrough such that the tip of the surgical drill bit extends past a bottom opening at a bottom region of the surgical drill sheath. Although a pedicle drill is shown for purposes of illustration and discussion, it will be appreciated that other types of surgical drills, and other medical devices and drills can alternatively be used with surgical system 10.

As shown in FIG. 1B, surgical drill sheath 100 can include a main body 110 through which the surgical drill bit 2 extends. Surgical drill sheath can also include a movable drill sheath insert 120 disposed within main body 110, and this movable drill sheath insert can also receive the surgical drill bit 2 therethrough. Movable drill sheath insert 120 can be part of an adjustment arrangement for surgical drill sheath 100, and as such can be raised or lowered within main body 110 to set a specific drill depth for surgical drill bit 2. This specific drill depth can correspond to the maximum distance that surgical drill bit 2 can extend past the bottom opening in main body 110. This maximum distance or drill depth can be achieved when a bottom surface 3 of surgical drill 1 contacts a mechanical hard stop proximate a top region of main body 110, which mechanical hard stop can be a top surface of movable drill sheath insert 120 as shown. When such contact is made between surgical drill bottom surface 3 and the top of movable drill sheath insert 120, the surgical drill 1 cannot move any further and surgical drill bit 2 has reached its drill depth. Raising or lowering movable drill sheath insert 120 can allow for a plurality of different specific drill depths, as set forth in greater detail below.

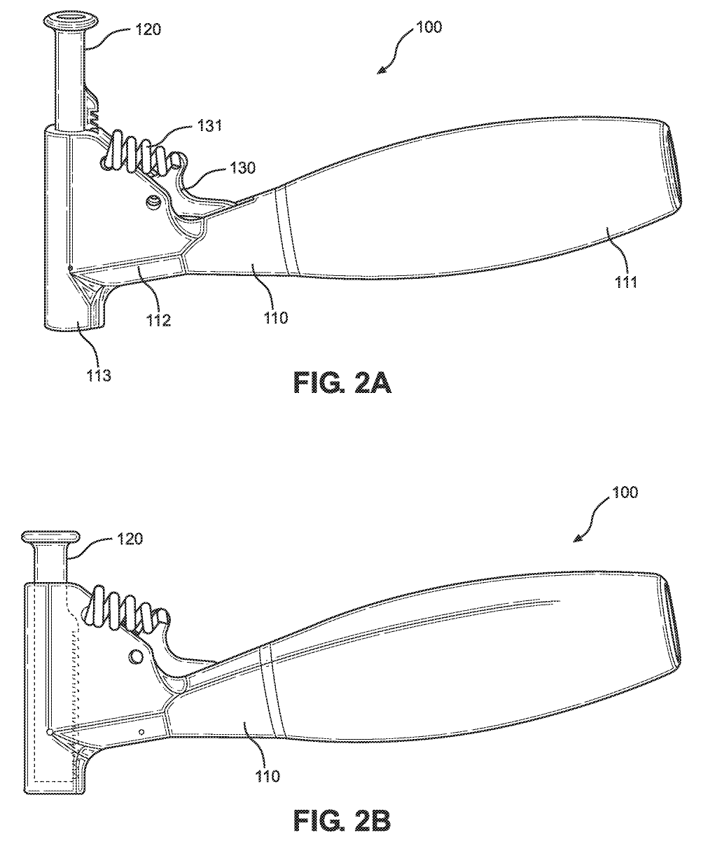

Turning next to FIGS. 2A and 2B, an example adjustable surgical drill sheath is shown in side elevation views with a raised insert and a lowered insert respectively. Surgical drill sheath 100, which can be considered an adjustable surgical drill sheath, can generally include a main body 110 and an adjustment arrangement configured to facilitate setting a specific drill depth. Main body 110 can include a handle 111, a midsection 112 having a top region and a bottom region, and a hollow region 113 between the top and bottom regions. Handle 111 can be configured to allow a surgeon or other operator to hold surgical drill sheath 100. In some arrangements, handle 111 can be separate from and coupled to main body 110. Main body 110 can be configured to receive a separate surgical drill bit through a top opening in the top region, through the hollow region 113, and through a bottom opening in the bottom region.

As noted above, the adjustment arrangement for surgical drill sheath 100 can include movable drill sheath insert 120. The adjustment mechanism can also include a latching mechanism configured to facilitate locking movable drill sheath insert 120 at a specific locked position relative to main body 110. This latching mechanism can be spring loaded and can include pivoting member 130 and spring 131, which spring can bias the latching mechanism to a locked position. The adjustment arrangement can include additional components and features, as set forth in greater detail below.

As shown in FIG. 2A, movable drill sheath insert 120 is at a raised position, such that the distance between the bottom opening at hollow region 113 and the top of the movable drill sheath insert (i.e., mechanical hard stop) is relatively large. This raised position for insert 120 results in a relatively short drill depth due the large distance between the hard stop and drill sheath bottom holding much of the drill bit within the insert and main body 110. Conversely, FIG. 2B shows movable drill sheath insert 120 is at a lowered position, such that the distance between the bottom of hollow region 113 and the top of the movable drill sheath insert is as small as possible. This lowered position results in the maximum possible drill depth for the surgical drill bit being used. Although fully raised and fully lowered positions are shown in FIGS. 2A and 2B for movable drill sheath insert 120, it will be understood that a plurality of intermediate positions are also possible, which can allow for greater drill depth control.

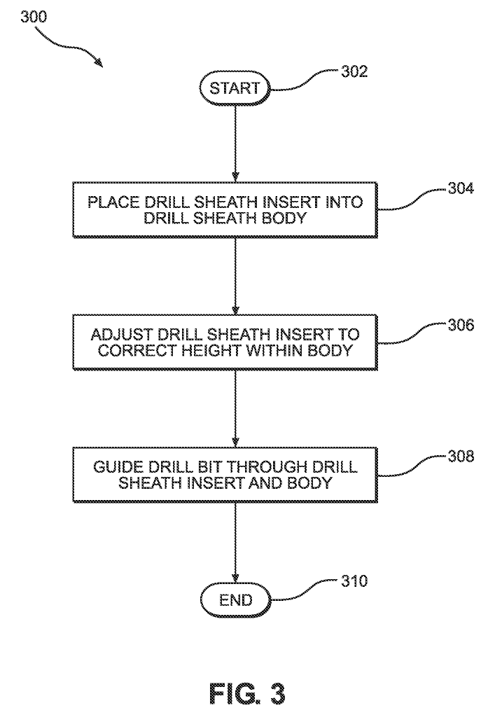

Continuing with FIG. 3 a flowchart of an example summary method of using a surgical drill sheath is provided. Summary method 300 can represent one broad aspect for overall methods of use for a surgical drill sheath, and it will be understood that various other steps, features, and details of such a broad aspect and overall methods of use are not provided here for purposes of simplicity. While the disclosed surgical drill sheath can be designed for use with a pedicle drill or other surgical drill as disclosed herein, it is contemplated that the surgical drill sheath can also be used with other objects and in other environments and applications.

After a start step 302, a first process step 304 can involve placing a drill sheath insert into a main body of a surgical drill sheath. This can involve a surgical drill sheath having a main body with a top region, a bottom region, and a hollow region between the top and bottom regions, and the drill sheath insert can be placed into the hollow region. The drill sheath insert can be movable, and as such can be raised and lowered within the hollow region. Step 304 can be optional in some situations, such as where a drill sheath insert is already located within the main body. Step 304 can be performed manually or automatically, such as where a separate robotic system can be configured to handle the surgical drill sheath and drill sheath insert.

At the following process step 306, the drill sheath insert can be adjusted to a correct height within the main body. This correct height can correspond to a specific drill depth that reflects the maximum distance that a separate surgical drill bit operated by a separate surgical drill can extend past a bottom opening in the bottom region of the main body. Step 306 can be manually or automatically performed, such as where a separate robotic system can be configured to handle the surgical drill sheath and drill sheath insert, such as to move and lock in place the drill sheath insert appropriately.

At subsequent process step 308, a separate surgical drill bit can be guided through the drill sheath insert and the main body of the surgical drill sheath. This can be done such that the surgical drill bit extends through the entire drill sheath insert as well as through a top opening in the top region, through the hollow region, and through the bottom opening in the bottom region of the main body. Step 308 can be manually or automatically performed, such as where a separate robotic system can be configured to handle a surgical drill and the surgical drill sheath, such as to guide a desired drill bit therethrough. The method can then end at end step 310.

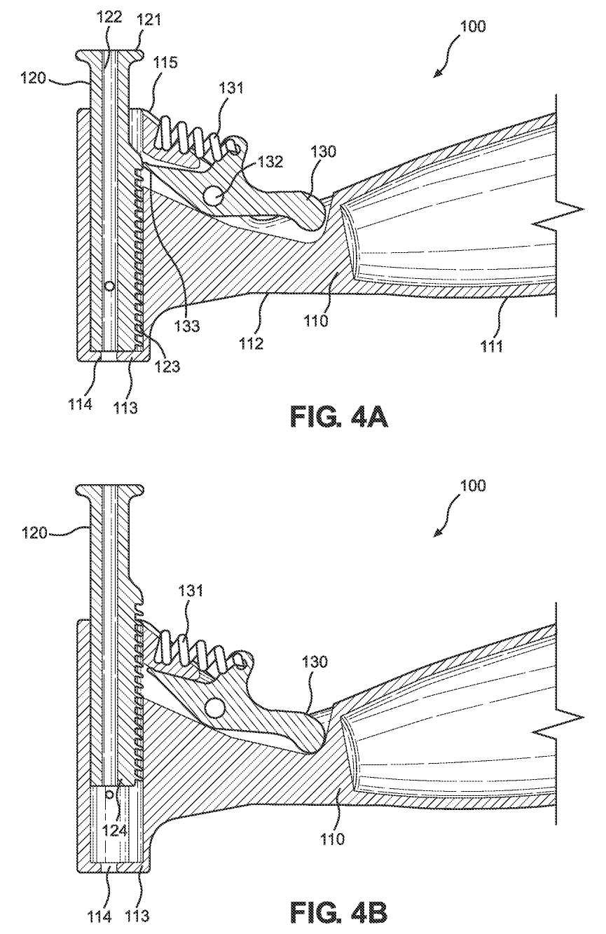

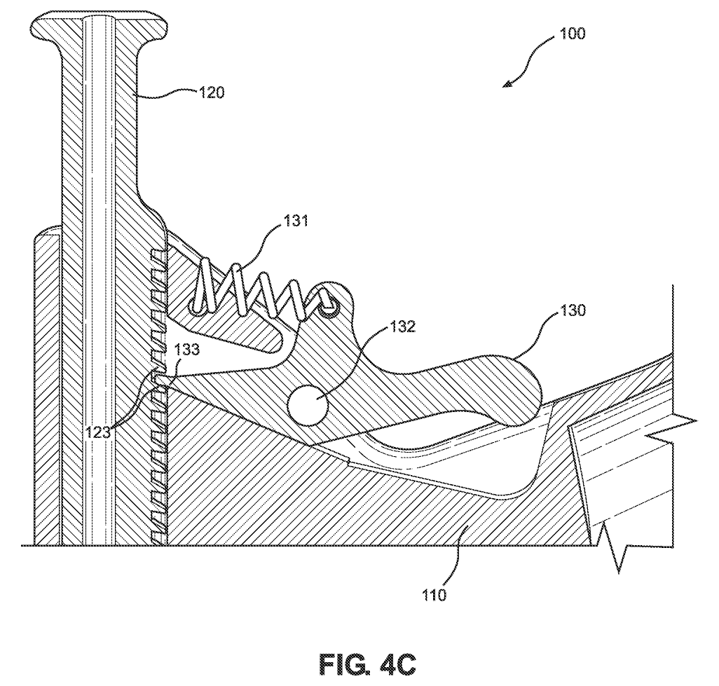

Moving now to FIGS. 4A through 4C, an adjustable region of an example adjustable surgical drill sheath is shown in side cross-section views with its movable drill sheath at different positions. FIG. 4A shows the adjustable surgical drill sheath with its movable drill sheath insert fully lowered and unlocked. Again, surgical drill sheath 100 can include a handle 111 integrally formed with or coupled to a main body 110 having a central region 112 and a hollow region 113 configured to accept a movable drill sheath insert 120 therein. Movable drill sheath insert 120 can include a top surface 121 that provides a mechanical hard stop for the bottom of a surgical drill to abut against when used in conjunction with surgical drill sheath 100. Movable drill sheath insert 120 can also include a cylindrical opening 122 configured to accept a separate surgical drill bit therethrough from its top surface 121 through to its bottom surface 124. The separate surgical drill bit can extend all the way through cylindrical opening 122 so that it is received through top opening 115 of main body 110 and exits the main body at bottom opening 114 at the bottom of hollow portion 113. Cylindrical opening 122 can have a diameter that corresponds to the diameter of a surgical drill bit to be used with movable drill sheath insert 120.

Movable drill sheath insert 120 can be one component of an adjustment arrangement configured to facilitate setting an exact drill depth. This adjustment arrangement can also have a latching mechanism that includes pivoting member 130, spring 131, pivot pin 132, and pawl 133 at one end of the pivoting member. Pawl 133 can be a locking mechanism configured to interact with a plurality of teeth or other suitable locking features 123 located along one side of movable drill sheath insert 120. In some arrangements, the latching mechanism can be spring loaded such that pawl 133 engages with locking features 123 to lock movable drill sheath insert 120 in place by default. The end of pivoting member 130 opposite pawl 133 can then be manually depressed to unlock the latching mechanism and enable movable drill sheath insert 120 to move up and down within hollow region 113. As shown in FIG. 4A, movable drill sheath insert 120 is fully lowered within hollow region 113 such that its bottom outer surface abuts against the inner surface of main body 110 at hollow region 113. Pivoting member 130 is also depressed, such that the latching mechanism is not engaged and movable drill sheath insert 120 is unlocked.

FIG. 4B illustrates the adjustable surgical drill sheath with its movable drill sheath insert partially raised and unlocked. As in the case of FIG. 4A, the latching mechanism is similarly not engaged such that movable drill sheath insert 120 is similarly unlocked and can be freely moved up and down. In some arrangements, drill sheath insert 120 can also be completely removed from main body 110 when the latching mechanism is unlocked. Unlike FIG. 4A, movable drill sheath insert 120 in FIG. 4B has been partially raised within hollow region 113 such that its bottom surface 124 is distanced apart from the inner bottom surface of the main body hollow region. This partially raised distance can correspond to a desired drill depth once a known drill bit is inserted through drill sheath insert 120 and main body 110.

FIG. 4C depicts the adjustable surgical drill sheath with its movable drill sheath insert partially raised and locked. Movable drill sheath insert 120 is partially raised similar to that which is shown in FIG. 4B, but the insert is locked into that exact raised position by way of the latching mechanism being active. As such, pawl 133 is engaged between teeth or other locking features 123 along an outer side of drill sheath insert 120 such that the insert is locked in place and cannot move up or down within hollow region 113. This configuration of the latching mechanism can be the default configuration due to spring 133 pulling pivoting member 130 such that it pivots about pivot pin 132 to force pawl 133 into locking features 123. Again, the latching mechanism can be unlocked from this configuration by manually pushing down on pivoting member 130 at the end opposite pawl 133, as will be readily appreciated.

In various embodiments, many different drill depth settings can be provided by way of many different locking positions using teeth or locking features 123 along the side of movable drill sheath insert 120. For example, a dozen or more different drill depth settings can be provided for some movable drill sheath inserts 120. Movable drill sheath insert 120 can have a plurality of different specific drill depths over a range from about 8 to 35 mm, for example, although other minimum and maximum drill depths are also possible. Movable drill sheath insert 120 can also be adjusted at set increments between the minimum and maximum positions, such as increments of 2 mm, for example, although other increments are also possible. In some arrangements, moveable drill sheath insert can have external markings along its outer surface that protrudes from the top hollow region 113 of main body 110. Such markings can reflect the drill depth to be expected for the lowest marking visible at a given setting. For example, a drill depth external marking for the drill sheath insert 120 shown in FIG. 2A can read “8 mm” (i.e., minimum drill depth), while a drill depth external marking for the movable drill sheath insert 120 shown in FIG. 2B can read “35 mm” (i.e., maximum drill depth). Other markings, drill depths, and arrangements are also possible.4

In some arrangements, movable drill sheath insert 120 can be configured to be removed completely from main body 110 and can even be replaced by a different movable drill sheath insert. For example, surgical drill sheath 100 can be configured to receive an alternative movable drill sheath insert (not shown) within the main body when the movable drill sheath insert 120 is removed. This can be done to allow for different drill bit sizes as well as different sets of drill depths. For example, an original movable drill sheath insert 120 and an alternative movable drill sheath insert can be configured for use with differently dimensioned surgical drill bits, such as drill bits having different diameters, different lengths, or both. Thus, an alternative or replacement movable drill sheath insert can have a cylindrical opening of a different diameter than that for cylindrical opening 122 of original movable drill sheath insert 120. In various embodiments, multiple different removable drill sheath inserts can be used with a given surgical drill sheath 100. For example, an overall system can include three different drill sheath inserts having cylindrical openings with diameters of about 2, 2.5, and 3 mm. Accordingly, surgical drill sheath 100 can be configured for use with separate surgical drill bits having diameters of about 2, 2.5, or 3 mm.

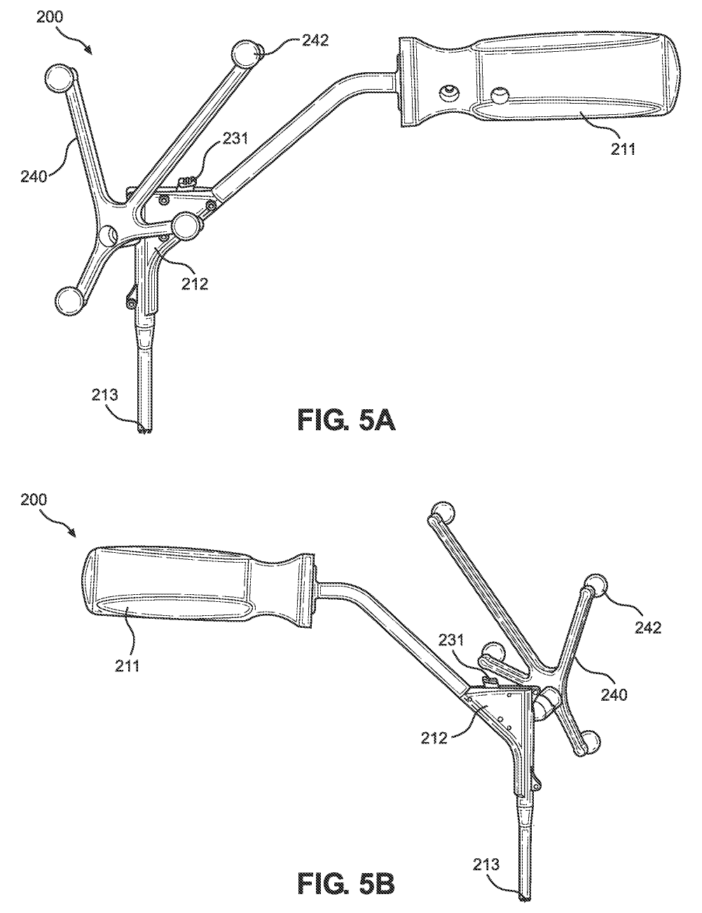

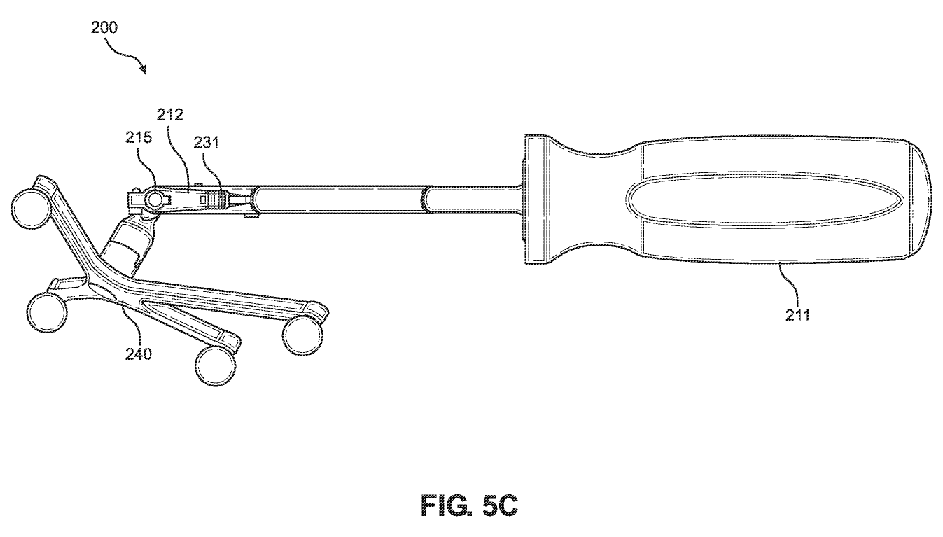

Continuing with FIGS. 5A through 5C, an example adjustable navigated surgical drill sheath is illustrated in side elevation, reverse side elevation, and top plan views respectively. Adjustable navigated surgical drill sheath 200 can be similar to surgical drill sheath 100 above in that it can include a handle 211, main body 212 having hollow region 213 and top opening 215 at an upper surface configured to receive a separate surgical drill bit therethrough, and adjustment arrangement 231 configured to facilitate setting a specific drill depth for the separate surgical drill bit. Unlike the foregoing embodiment, adjustable navigated surgical drill sheath 200 can also include a fiducial marker array 240 having multiple fiducial markers 242 configured to facilitate tracking in an augmented reality environment.

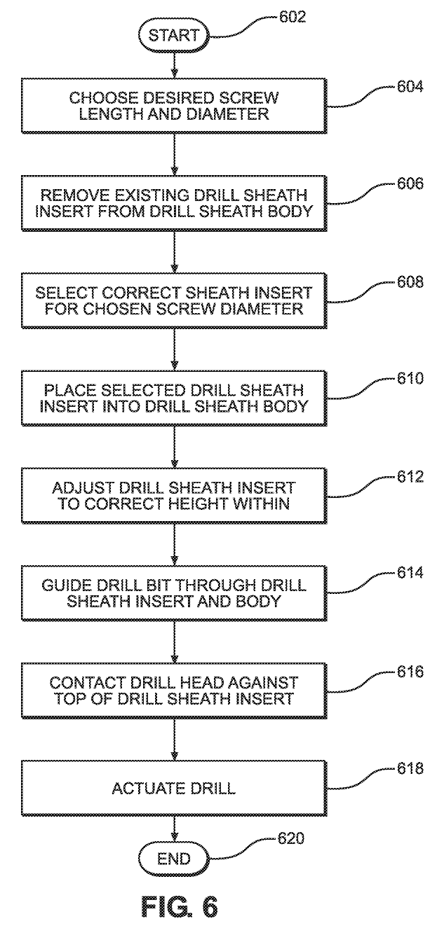

Lastly, FIG. 6 illustrates a flowchart of an example detailed method of using a surgical drill sheath. Detailed method 600 can represent one possible way of using a surgical drill sheath, and it will be understood that various other steps, features, and details of such a detailed method are not provided here for purposes of simplicity. Detailed method 600 can also include some or all steps and details of summary method 300 above, as will be readily appreciated. After a start step 602, a first process step 604 can involve choosing a desired surgical screw length and diameter. When using a pedicle drill, for example, drill holes are made for the purpose of inserting pedicle screws. Depending on the patient, different pedicle screws can be used, the selection of which can be chosen at step 604. Step 604 can be manually or automatically performed, such as where a computing system can be configured to choose screw length and diameter based on patient attributes.

A following process step 606 can involve removing an existing drill sheath insert from the main body of the surgical drill sheath. This step can be optional and need not be performed if the existing drill sheath insert is actually the correct one for the desired drill depth. Step 606 can be performed manually or automatically in some cases, such as where a separate robotic system can be configured to handle the surgical drill sheath and drill sheath insert.

At subsequent process step 608, a correct drill sheath insert can be selected based on the chosen screw diameter. The correct insert can have a cylindrical opening with a diameter that corresponds to the correct drill bit, which can in turn correspond to the chosen screw diameter. Again, such diameter choices can be 2 mm, 2.5 mm, or 3 mm, although other size diameters are also possible. Step 608 can be manually or automatically performed, such as where a computing system can be configured to select the correct drill sheath insert based on the chosen screw length and diameter.

The next process step 610 can be identical or substantially similar to step 304 above, and can involve placing a drill sheath insert into a main body of a surgical drill sheath. This can involve a surgical drill sheath having a main body with a top region, a bottom region, and a hollow region, and the drill sheath insert can be placed into the hollow region. The drill sheath insert can be movable, and as such can be raised and lowered within the hollow region. Step 610 can be performed manually or automatically, such as where a separate robotic system can be configured to handle the surgical drill sheath and drill sheath insert.

At a following process step 612, which can be identical or substantially similar to step 306 above, the drill sheath insert can be adjusted to a correct height within the main body. This correct height can correspond to a specific drill depth that reflects the maximum distance that a separate surgical drill bit operated by a separate surgical drill can extend past a bottom opening in the bottom region of the main body. Step 612 can be manually or automatically performed, such as where a separate robotic system can be configured to handle the surgical drill sheath and drill sheath insert, such as to move and lock in place the drill sheath insert appropriately.

At subsequent process step 614, which can be identical or substantially similar to step 308 above, a separate surgical drill bit can be guided through the drill sheath insert and the main body of the surgical drill sheath. This can be done such that the surgical drill bit extends through the entire drill sheath insert as well as through a top opening in the top region, through the hollow region, and through the bottom opening in the bottom region of the main body. Step 614 can be manually or automatically performed, such as where a separate robotic system can be configured to handle a surgical drill and the surgical drill sheath, such as to guide a desired drill bit therethrough.

At the next process step 616, the drill head or body can be contacted against a mechanical hard stop on the surgical drill sheath. Such a hard stop can be the top of the drill sheath insert as the insert is set at the correct height. Step 616 can be manually or automatically performed, such as where the natural physical consequences of using the surgical drill and the surgical drill sheath with the insert set at the correct height results in the drill head or body moving downward to contact the mechanical hard stop.

At process step 618 the surgical drill can be actuated. This can result in a hole being drilled at or about the exact drill depth desired. Step 618 can be manually or automatically performed, such as where a separate robotic system can be configured to handle and operate the surgical drill to drill the hole as desired. The method can then end at end step 620.

For foregoing method 600, it will be appreciated that not all process steps are necessary, and that other process steps may be added in some arrangements. For example, some or all of the steps can be repeated where multiple screws are to be used such that multiple holes are to be drilled. Furthermore, the order of steps may be altered in some cases, and some steps may be performed simultaneously. For example, step 618 may be performed before step 616. As another example, steps 610 and 612 can be performed simultaneously. Although known process steps are provided for the various techniques in detailed method 600, it will be appreciated that any other suitable similar method for using a surgical drill sheath can also be used. Other variations and extrapolations of the disclosed methods will also be readily appreciated by those of skill in the art.

Although the foregoing disclosure has been described in detail by way of illustration and example for purposes of clarity and understanding, it will be recognized that the above described disclosure may be embodied in numerous other specific variations and embodiments without departing from the spirit or essential characteristics of the disclosure. Certain changes and modifications may be practiced, and it is understood that the disclosure is not to be limited by the foregoing details, but rather is to be defined by the scope of the appended claims.

What is claimed is:

1. A surgical drill sheath, comprising:
a handle configured to allow an operator to hold the surgical drill sheath;
a main body coupled to the handle, the main body having a top region, a bottom region, and a hollow region between the top and bottom regions, wherein the main body is configured to receive a separate surgical drill bit through a top opening in the top region, through the hollow region, and through a bottom opening in the bottom region; and
an adjustment arrangement configured to facilitate setting a specific drill depth for the separate surgical drill bit received through the main body, the specific drill depth corresponding to the maximum distance that the separate surgical drill bit can extend past the bottom opening, wherein the adjustment arrangement allows for a plurality of different specific drill depths.

2. The surgical drill sheath of claim 1, wherein the adjustment mechanism includes a mechanical hard stop proximate the top region, the mechanical hard stop being configured to abut against a bottom surface of a separate surgical drill operating the separate surgical drill bit when the specific drill depth is reached.

3. The surgical drill sheath of claim 2, wherein the adjustment mechanism is configured to facilitate adjusting a distance between the mechanical hard stop and the bottom opening.

4. The surgical drill sheath of claim 2, wherein the separate surgical drill is a pedicle drill.

5. The surgical drill sheath of claim 1, wherein the adjustment mechanism includes a movable drill sheath insert located within the hollow region and configured to be moved up and down within the hollow region to facilitate setting the specific drill depth.

6. The surgical drill sheath of claim 5, wherein the movable drill sheath insert includes a plurality of locking features along an outer surface thereof, and wherein the adjustment mechanism further includes a latching mechanism configured to interact with the plurality of locking features to lock the movable drill sheath insert at a specific locked position relative to the main body to facilitate setting the specific drill depth.

7. The surgical drill sheath of claim 6, wherein the latching mechanism includes a pivoting member having a pawl that engages with the plurality of locking features to lock the movable drill sheath insert at the specific locked position.

8. The surgical drill sheath of claim 7, wherein the pivoting member is spring loaded to lock the movable drill sheath insert at a specific locked position, and wherein the pivoting member further includes a manual release feature to overcome the spring loaded locked position and allow the movable drill sheath insert to be moved.

9. The surgical drill sheath of claim 5, wherein the movable drill sheath insert includes a cylindrical opening therethrough configured to accept the separate surgical drill bit therethrough and having a diameter that corresponds to the diameter of the separate surgical drill bit.

10. The surgical drill sheath of claim 5, wherein the movable drill sheath insert is configured to be removed from the main body.

11. The surgical drill sheath of claim 5, wherein the surgical drill sheath is configured to receive an alternative movable drill sheath insert within the main body when the movable drill sheath insert is removed, and wherein the movable drill sheath insert and alternative movable drill sheath insert are configured for use with differently dimensioned separate surgical drill bits.

12. The surgical drill sheath of claim 1, wherein the surgical drill sheath is configured for use with separate surgical drill bits having diameters of about 2, 2.5, or 3 mm.

13. The surgical drill sheath of claim 1, wherein the plurality of different specific drill depths range from about 8 to 35 mm.

14. The surgical drill sheath of claim 1, wherein the plurality of different specific drill depths can be adjusted by increments of about 2 mm.

15. The surgical drill sheath of claim 1, further comprising:
a plurality of fiducial markers coupled to the main body, wherein the plurality of fiducial markers are configured to facilitate use of the surgical drill sheath as a navigated device in an augmented reality environment.

16. An adjustable navigated surgical drill sheath, comprising:
a handle configured to allow an operator to hold the adjustable navigated surgical drill sheath;
a main body coupled to the handle, the main body having a top region, a bottom region, and a hollow region between the top and bottom regions, wherein the main body is configured to receive a separate surgical drill bit through a top opening in the top region, through the hollow region, and through a bottom opening in the bottom region;
an adjustment arrangement configured to facilitate setting a specific drill depth for the separate surgical drill bit received through the main body, the specific drill depth corresponding to the maximum distance that the separate surgical drill bit can extend past the bottom opening, the adjustment arrangement allowing for a plurality of different specific drill depths, wherein the adjustment mechanism includes a removable drill sheath insert having a plurality of locking features and configured to be moved up and down within the hollow region and a latching mechanism having a spring loaded pivoting member with a pawl configured to interact with the plurality of locking features to lock the removable drill sheath insert at a specific locked position relative to the main body to facilitate setting the specific drill depth; and
a plurality of fiducial markers coupled to the main body, wherein the plurality of fiducial markers are configured to facilitate use of the surgical drill sheath as a navigated device in an augmented reality environment.

17. The adjustable navigated surgical drill sheath of claim 16, wherein the removable drill sheath insert includes a cylindrical opening therethrough having a diameter that corresponds to the separate surgical drill bit diameter, and wherein the removable drill sheath insert is configured to be replaced with an alternative removable drill sheath insert having a cylindrical opening of a different diameter that corresponds to a separate alternative surgical drill bit diameter.

18. The adjustable navigated surgical drill sheath of claim 16, wherein the adjustable navigated surgical drill sheath is configured for use with separate pedicle drill bits having diameters of about 2, 2.5, or 3 mm, wherein the plurality of different specific drill depths range from about 8 to 35 mm, and wherein the plurality of different specific drill depths can be adjusted by increments of about 2 mm.

19. A method of using a surgical drill sheath, the method comprising:
placing a drill sheath insert into a main body of the surgical drill sheath, the main body having a top region, a bottom region, and a hollow region between the top and bottom regions;
adjusting the drill sheath insert to a correct height within the main body, wherein the correct height corresponds to a specific drill depth that reflects the maximum distance that a separate surgical drill bit operated by a separate surgical drill can extend past a bottom opening in the bottom region of the main body; and
guiding a separate surgical drill bit through the drill sheath insert and the main body of the surgical drill sheath such that the surgical drill bit extends through the entire drill sheath insert as well as through a top opening in the top region, through the hollow region, and through the bottom opening in the bottom region of the main body.

20. The method of claim 19, wherein the separate surgical drill is a pedicle drill and further comprising the steps of:
choosing a desired length and diameter for one or more pedicle screws to be used in a subsequent pedicle operation;
remove an existing drill sheath insert from the main body of the surgical drill sheath;
select the drill sheath insert to be placed into the main body based on the chosen pedicle screw diameter;
contact a drill head of the pedicle drill against a top of the drill sheath insert adjusted within the main body; and
actuate the pedicle drill.
